(19) United States
(12) Patent Application Publication
(10) Pub. No.: US 2021/0401481 A1
(43) Pub. Date: Dec. 30, 2021

(54) INDUCTIVELY HEATED PERFORATOR WITH SUCTION

(71) Applicant: Covidien LP, Mansfield, MA (US)

(72) Inventor: Richard L. Croft, Mead, CO (US)

(21) Appl. No.: 17/330,522

(22) Filed: May 26, 2021

Related U.S. Application Data
(60) Provisional application No. 63/043,927, filed on Jun. 25, 2020.

(51) Int. Cl. A61B 18/08; A61B 34/35

(52) U.S. Cl. CPC A61B 18/082; A61B 2017/00876; A61B 2218/008; A61B 34/35

(57) ABSTRACT

A perforator system includes a tube, a solenoid coil, and a heating shaft. The tube defines a longitudinal axis and a lumen that extends along the longitudinal axis. The solenoid coil is supported within the tube about the longitudinal axis and defines a central passage therethrough. The heating shaft is received in the central passage and is positioned to inductively couple to the solenoid coil. The heating shaft is configured to dissipate an amount of heat sufficient to treat tissue received in the lumen when electrical energy is conducted through the solenoid coil.

### CROSS REFERENCE TO RELATED APPLICATIONS

This application claims the benefit of U.S. Provisional Application Ser. No. 63/043,927, filed Jun. 25, 2020, the entire contents of which are incorporated by reference herein.

### TECHNICAL FIELD

This disclosure relates to perforator devices, and more particularly, to perforator devices for effectuating small perforations in tissue such as during an enterotomy procedure.

### BACKGROUND

Enterotomy is the surgical incision into an intestine such as during exploratory laparotomies or hernia repair. An enterotomy can be done to remove an obstruction or foreign body from the intestine. Perforation of membranes, such as membranes of the intestine during an enterotomy, are typically blind and require puncturing a surface of the membrane by pressing into the membrane without causing structural damage behind the membrane.

### SUMMARY

In accordance with an aspect of this disclosure, a perforator system includes a tube, a solenoid coil, and a heating shaft. The tube defines a longitudinal axis and a lumen that extends along the longitudinal axis. The solenoid coil is supported within the tube about the longitudinal axis and defines a central passage therethrough. The heating shaft is received in the central passage and is positioned to inductively couple to the solenoid coil. The heating shaft is configured to dissipate an amount of heat sufficient to treat tissue received in the lumen when electrical energy is conducted through the solenoid coil.

In aspects, the perforator system may further include a vacuum source in fluid communication the lumen. The vacuum source may be configured to apply suction force through the lumen to draw tissue, smoke, and/or debris into the tube.

In aspects, the heating shaft may include metallic material.

In aspects, the perforator system may further include an electrosurgical energy source in electrical communication with the solenoid coil.

In aspects, the heating shaft may be disposed in radially spaced relation with the solenoid coil.

In aspects, the tube may include non-conductive material.

In aspects, inductive energy from the solenoid coil may cause an outer surface of the heating shaft to dissipate the amount of heat sufficient to treat tissue.

In aspects, the heating shaft may extend to a pointed or sharpened tip.

In aspects, the heating shaft may define a distal pocket therein.

In aspects, the heating shaft may define a longitudinal lumen at least partially therethrough.

According to another aspect, a robotic perforator system includes a robotic arm and a perforator supported on the robotic arm. The perforator includes a tube defining a longitudinal axis and a lumen that extends along the longitudinal axis. A solenoid coil is supported within the tube about the longitudinal axis and defines a central passage therethrough. A heating shaft is received in the central passage and positioned to inductively couple to the solenoid coil. The heating shaft is configured to dissipate an amount of heat sufficient to treat tissue received in the lumen when electrical energy is conducted through the solenoid coil.

According to yet another aspect, a handheld perforator includes a handle assembly, a tube, a solenoid coil, and a heating shaft. The tube extends distally from the handle assembly. The tube defines a longitudinal axis and a lumen that extends along the longitudinal axis. The solenoid coil is supported within the tube about the longitudinal axis and defines a central passage therethrough. The heating shaft is received in the central passage and is positioned to inductively couple to the solenoid coil. The heating shaft is configured to dissipate an amount of heat sufficient to treat tissue received in the lumen when electrical energy is conducted through the solenoid coil.

Other aspects, features, and advantages will be apparent from the description, the drawings, and the claims that follow.

### BRIEF DESCRIPTION OF THE DRAWINGS

The accompanying drawings, which are incorporated in and constitute a part of this specification, illustrate aspects of this disclosure and, together with a general description of this disclosure given above, and the detailed description given below, serve to explain the principles of this disclosure, wherein:

FIG. 1 is a perspective view of a robotic system supporting a robotic perforator device in accordance with the principles of this disclosure;

FIG. 2 is an enlarged, perspective view of a proximal portion of the robotic perforator device of FIG. 1;

FIG. 3 is an enlarged, perspective view of a distal portion of the robotic perforator device of FIG. 1;

FIG. 4 is a perspective view of a solenoid coil of the robotic perforator device of FIG. 1;

FIG. 5 is a perspective view of a distal portion of an illustrative heating element of the robotic perforator device of FIG. 1;

FIG. 6 is a perspective view of a distal portion of another illustrative heating element of the robotic perforator device of FIG. 1; and

FIG. 7 is a perspective view of a hand-held robotic perforator device in accordance with the principles of this disclosure.

### DETAILED DESCRIPTION

Aspects of this disclosure are described in detail with reference to the drawings, in which like reference numerals designate identical or corresponding elements in each of the several views. As used herein, the term “distal” refers to that portion of structure farther from the user, while the term “proximal” refers to that portion of structure, closer to the user. As used herein, the term “clinician” refers to a doctor, nurse, or other care provider and may include support personnel.

In the following description, well-known functions or constructions are not described in detail to avoid obscuring the present disclosure in unnecessary detail.

Robotic surgical systems have been used in minimally invasive medical procedures and can include robotic arm assemblies. Such procedures may be referred to as what is commonly referred to as “Telesurgery.” Some robotic arm assemblies include one or more robot arms to which surgical instruments can be coupled. Such surgical instruments include, for example, electrosurgical forceps, cutting instruments, staplers, graspers, electrocautery devices, or any other endoscopic or open surgical devices. Prior to or during use of the robotic surgical system, various surgical instruments can be selected and connected to the robot arms for selectively actuating end effectors of the connected surgical instruments. Some of these surgical instruments utilize electrical energy, for example, to effectuate electrocautery.

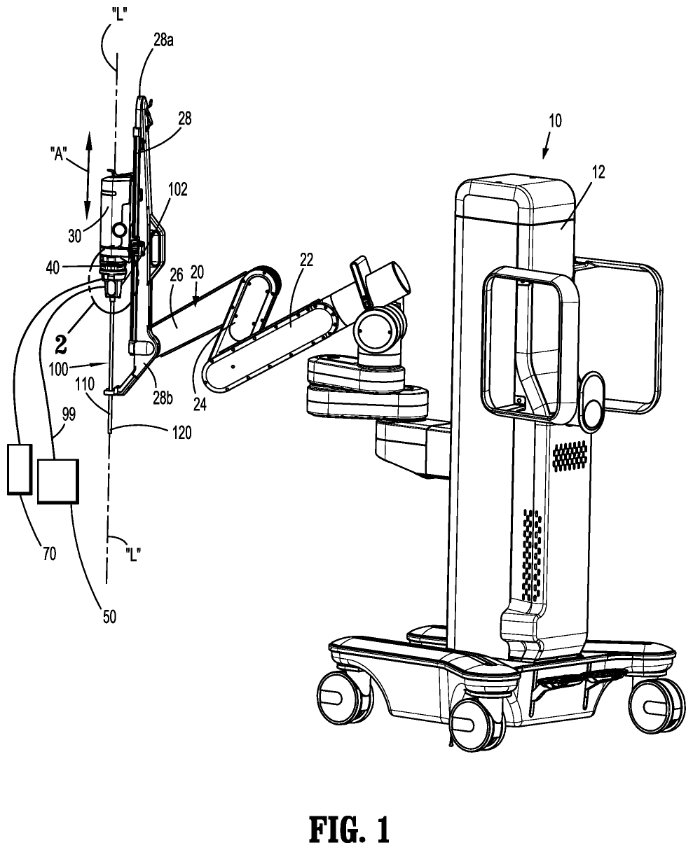

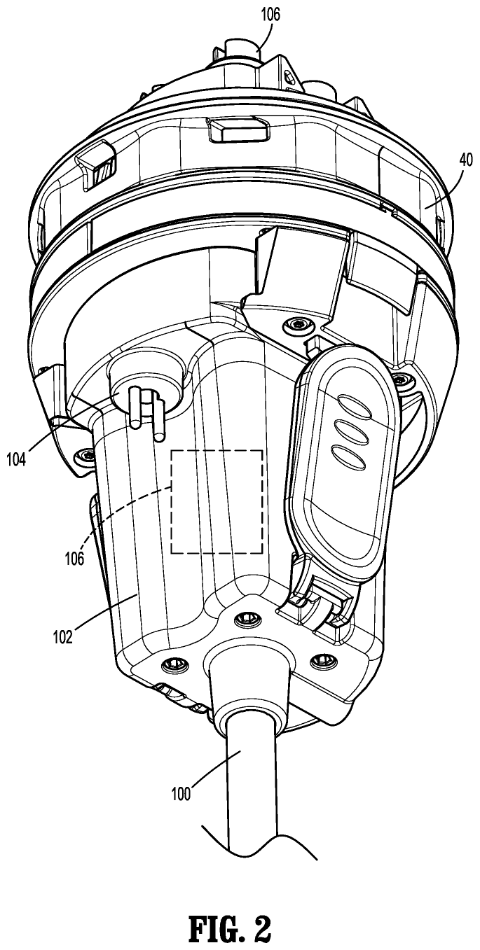

With reference to FIGS. 1 and 2, a robotic surgical system, such as the robotic perforator system 10 illustrated in FIG. 1, includes a robotic arm assembly 20 that supports a surgical instrument, such as a perforator 100, for effectuating a surgical procedure (e.g., an enterotomy), an instrument drive unit 30 that imparts driving force to perforator 100, and a sterile interface module 40 that enables a proximal housing assembly 102 of perforator 100 to interface with instrument drive unit 30. This interface advantageously maintains sterility, provides a means to transmit electrical communication between robotic enterotomy system 10 and perforator 100, provides a means for transferring torque (e.g., rotational force) from robotic enterotomy system 10 (e.g., IDU 30) to perforator 100 for performing a function (e.g., sealing, cutting, manipulating, etc.) with perforator 100 and/or provides a means to selectively attach/remove perforator 100 to robotic enterotomy system 10 (e.g., for rapid instrument exchange). For a more detailed description of similar sterile interface modules and components thereof, reference can be made to WO2017205308 by Zemlock et al., the entire contents of which are incorporated by reference herein.

Robotic enterotomy system 10 further includes an energy source such as an electrosurgical generator 50 that couples to perforator 100 and/or any number of other surgical instruments (e.g., an electrosurgical probe or an electrocautery blade—not shown) via an electrosurgical cable 99 and a connector assembly 104 supported by sterile interface module 40 and/or proximal housing assembly 102 of perforator 100. For a more detailed description of one example of an electrosurgical generator, reference can be made to U.S. Pat. No. 8,784,410, the entire contents of which are incorporated by reference herein. For a more detailed description of one example of connector assembly 104, reference can be made to U.S. Patent Application No. 62/823,036, filed Mar. 25, 2019, and entitled “Robotic Surgical Systems with Electrical Switch of Instrument Attachment,” the entire contents of which are incorporated by reference herein. For a more detailed description of one example of an electrocautery blade, reference can be made to U.S. Pat. No. 8,128,622 or 8,460,289, the entire contents of each of which are incorporated herein by reference.

Robotic enterotomy system 10 employs various robotic elements to assist the clinician and allow remote operation (or partial remote operation) of surgical instrumentation such as perforator 100. Various robotic arms, gears, cams, pulleys, electric and mechanical motors, etc. may be employed for this purpose and may be designed with robotic enterotomy system 10 to assist the clinician during the course of an operation or treatment, and which can be included with, and/or part of one or more drive mechanisms 106 of perforator 100, sterile interface module 40, and/or instrument drive unit 30. Such robotic systems may include remotely steerable systems, automatically flexible surgical systems, remotely flexible surgical systems, remotely articulating surgical systems, wireless surgical systems, modular or selectively configurable remotely operated surgical systems, etc.

Robotic enterotomy system 10 includes a medical workstation (not shown) that may be employed with one or more consoles positioned next to the operating theater or located in a remote location. In this instance, one team of clinicians may prep the patient for surgery and configure robotic enterotomy system 10 with perforator 100 while another clinician (or group of clinicians) remotely controls perforator 100 via the one or more consoles. As can be appreciated, a highly skilled clinician may perform multiple operations in multiple locations without leaving his/her remote console. This can be economically advantageous and a benefit to the patient or a series of patients. For a detailed description of exemplary medical workstations and/or components thereof, reference may be made to U.S. Pat. No. 8,828,023 and PCT Application Publication No. WO2016/025132, the entire contents of each of which are incorporated by reference herein.

With continued reference to FIG. 1, robotic arm assembly 20 of robotic surgical system 10 includes a cart 12 having robotic arms 22, 24, 26 that are pivotally coupled together and movable together and/or relative to one another and cart 12. Robotic arm 26 is coupled to a slide rail 28 that supports instrument drive unit (“IDU”) 30 and sterile interface module 40 for operating perforator 100. IDU 30 defines a longitudinal axis “L” and is slidably supported on slide rail 28 and selectively axially movable along longitudinal axis “L,” as indicated by arrows “A,” between a proximal position adjacent a proximal end portion 28a of slide rail 28, and a distal position adjacent a distal end portion 28b of slide rail 28.

Robotic surgical system 10 can be in the form of an electrosurgical enterotomy system. In general, components of the electrosurgical enterotomy system can be used to effectuate an enterotomy.

With reference now to FIGS. 1-4, perforator 100 of robotic perforator system 10 includes an elongated shaft assembly 110 that extends from proximal housing assembly 102 to an end effector 120 at a distal end of elongated shaft assembly 110. Elongated shaft assembly 110 is coaxial with longitudinal axis “L.”

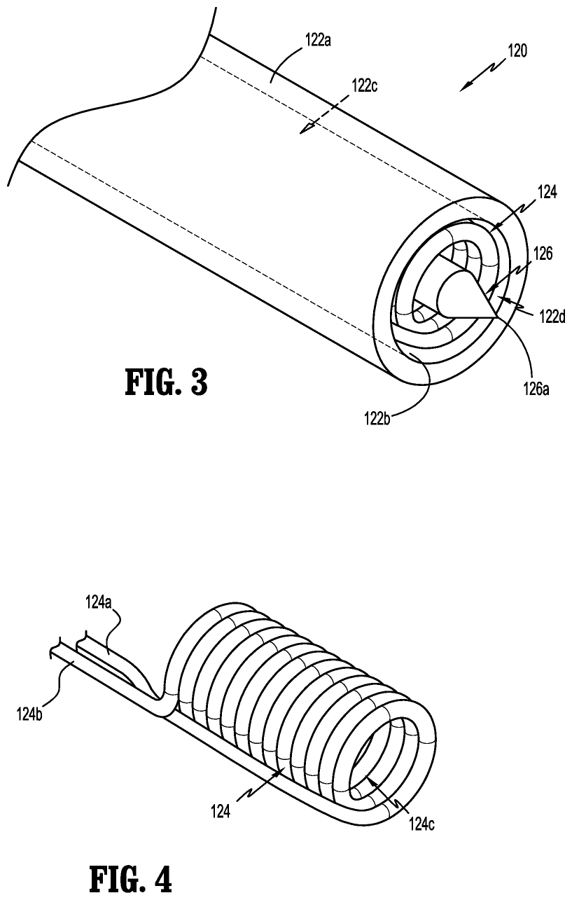

As seen in FIG. 3, end effector 120 of perforator 100 includes an outer tube 122 having an outer surface 122a and an inner surface 122b. Outer tube 122 may include any suitable non-conductive material such polymeric material. Inner surface 122b of outer tube 122b defines a lumen 122c that is disposed in fluid communication with a vacuum source 70 of robotic system 10 to enable suction of tissue into a distal opening 122d of lumen 122c and proximally through lumen 122c toward vacuum source 70. End effector 120 supports a solenoid coil 124, which may have number of windings that spiral around longitudinal axis “L,” mounted to or adjacent inner surface 122b of outer tube 122b. Solenoid coil 124 includes first and second end portions 124a, 124b that are electrically coupled to the electrosurgical energy source 99 of robotic system 10 to enable electrical energy to be conducted through solenoid coil 124. Solenoid coil 124 further defines a central passage 124c that is concentric with lumen 122c and positioned to receive a heating shaft 126 therethrough. The heating shaft 126 is positioned in radially spaced relation to solenoid coil 124, but sufficiently close to receive inductive energy from solenoid coil 124 when electrical energy is conducted through solenoid coil 124 (e.g., around heating shaft 126). Heating shaft 126 is formed of any suitable electrically conductive material, such as a metallic material, and is configured to inductively couple to solenoid coil 124 to enable to heating shaft 126 to heat up via induction from electrical energy received from solenoid coil 124. Heating shaft 126 may be at least partially solid and/or hollow. Heating shaft 126 extends to a pointed or sharpened distal tip 126a to enable tissue piercing.

In use, the distal end of end effector 120 is disposed adjacent to tissue to be perforated and the vacuum source 70 is activated to cause suction through end effector 120 to draw the tissue (e.g., tissue membrane) into lumen 122c of end effector 120 and away from underlying tissue or structures to prevent additional tissue damage. Electrical energy (e.g., current) is conducted through solenoid coil 124 to inductively heat heating shaft 126 to a temperature sufficient to cause the tissue drawn into the lumen 122c to be treated (e.g., cauterization) so as to create a perforation in the tissue. Any resulting debris and/or smoke is evacuated via the suction, through the lumen 122c toward the vacuum source 70. This advantageously helps to prevent spreading of any cancerous cells associated with the treated tissue.

In aspects, end effector 120 can include ferromagnetic materials, which may be coated thereon. In some aspects, end effector 120 may include or be operatively coupled to one or more sensors, such as temperature sensors to control a temperature of end effector 120. In aspects, end effector 120 can be heated based on a Curie point temperature of one or materials thereof.

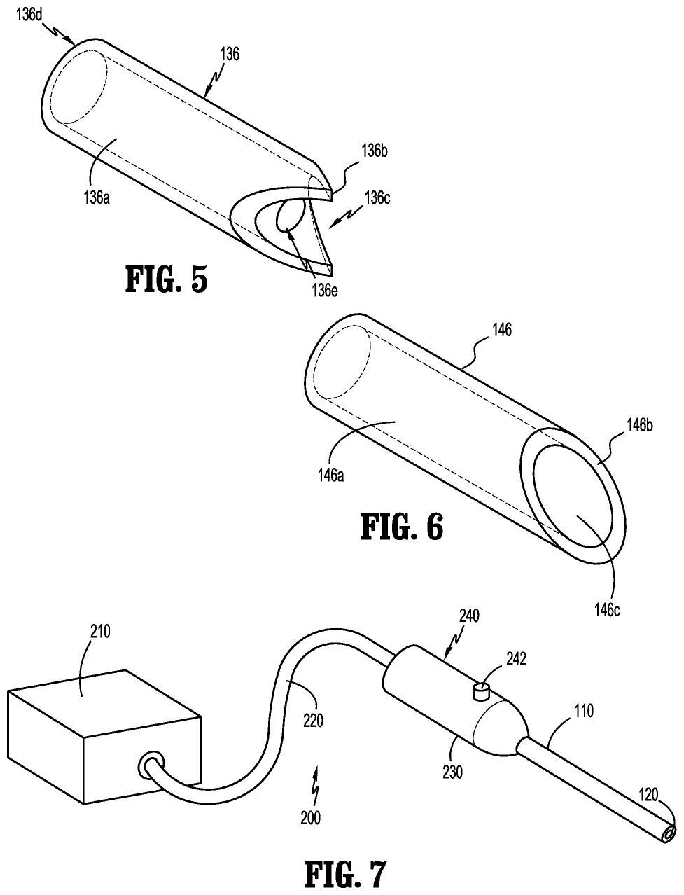

With reference to FIG. 5, another heating shaft for use in devices in accordance with the present disclosure, generally referred to as heating shaft 136, includes a tubular body 136a that extends to a pointed distal end 136b and defines a distal pocket 136c therein. Distal pocket 136c may have a C-shaped profile that tapers distally to pointed distal end 136b and opens distally and laterally through tubular body 136a. Tubular body 136a may define a longitudinal lumen or bore 136d that extends at least partially therethrough. Lumen or bore 136d may be in fluid communication with distal pocket 136c via an opening 136e. In aspects, distal pocket 136c partitioned from lumen or bore 136d (e.g., devoid of opening 136e).

As seen in FIG. 6, another heating shaft for use in devices in accordance with the present disclosure, generally referred to as heating shaft 146, includes a tubular body 146a that extends to a pointed tip 146b that may be beveled. Tubular body 146a defines a central longitudinal lumen 146c therethrough.

As illustrated in FIG. 7, although detailed herein with respect to a robotic system, the disclosed perforators can be provided as manual and/or hand-held instruments or systems such as perforator system 200 including a vacuum source 210, a conduit 220 extending from vacuum source 210 and a perforator 230 in fluid communication with the vacuum source 210 via conduit 220. Perforator 230 includes a handle assembly 240 and an elongated shaft assembly 110 extending distally from handle assembly 240 to end effector 120. Handle assembly 240 includes any number of actuators 242 for selectively activating end effector 120 and/or vacuum source 210. Handle assembly 210 supports any number of mechanical and/or electrical components (e.g., power source, circuitry, controllers, switches, circuit board, resistors, capacitors, inductors, chips, etc.) to operate end effector 120 similar to that detailed above with respect to robotic system 10.

Securement of any of the components of the disclosed devices may be effectuated using known securement techniques such welding, crimping, gluing, fastening, etc.

Persons skilled in the art will understand that the structures and methods specifically described herein and shown in the accompanying figures are non-limiting exemplary aspects, and that the description, disclosure, and figures should be construed merely as exemplary of particular aspects. It is to be understood, therefore, that this disclosure is not limited to the precise aspects described, and that various other changes and modifications may be effectuated by one skilled in the art without departing from the scope or spirit of the disclosure. Additionally, the elements and features shown or described in connection with certain aspects may be combined with the elements and features of certain other aspects without departing from the scope of this disclosure, and that such modifications and variations are also included within the scope of this disclosure. Accordingly, the subject matter of this disclosure is not limited by what has been particularly shown and described.

What is claimed is:

1. A perforator system comprising:
a tube defining a longitudinal axis and a lumen that extends along the longitudinal axis;
a solenoid coil supported within the tube about the longitudinal axis and defining a central passage therethrough; and
a heating shaft received in the central passage and positioned to inductively couple to the solenoid coil, the heating shaft configured to dissipate an amount of heat sufficient to treat tissue received in the lumen when electrical energy is conducted through the solenoid coil.

2. The perforator system of claim 1, further comprising a vacuum source in fluid communication the lumen and configured to apply suction force through the lumen to draw tissue, smoke, and/or debris into the tube.

3. The perforator system of claim 1, wherein the heating shaft includes metallic material.

4. The perforator system of claim 1, further comprising an electrosurgical energy source in electrical communication with the solenoid coil.

5. The perforator system of claim 1, wherein the heating shaft is disposed in radially spaced relation with the solenoid coil.

6. The perforator system of claim 1, wherein the tube includes non-conductive material.

7. The perforator system of claim 1, wherein inductive energy from the solenoid coil causes an outer surface of the heating shaft to dissipate the amount of heat sufficient to treat tissue.

8. The perforator system of claim 1, wherein the heating shaft extends to a pointed or sharpened tip.

9. The perforator system of claim 8, wherein the heating shaft defines a distal pocket therein.

10. The perforator system of claim 8, wherein the heating shaft defines a longitudinal lumen at least partially therethrough.

11. A robotic perforator system comprising:
a robotic arm; and
a perforator supported on the robotic arm, the perforator including:
a tube defining a longitudinal axis and a lumen that extends along the longitudinal axis;
a solenoid coil supported within the tube about the longitudinal axis and defining a central passage therethrough; and
a heating shaft received in the central passage and positioned to inductively couple to the solenoid coil, the heating shaft configured to dissipate an amount of heat sufficient to treat tissue received in the lumen when electrical energy is conducted through the solenoid coil.

12. The robotic perforator system of claim 11, further comprising a vacuum source in fluid communication the lumen and configured to apply suction force through the lumen to draw tissue, smoke, and/or debris into the tube.

13. The robotic perforator system of claim 11, wherein the heating shaft includes metallic material.

14. The robotic perforator system of claim 11, further comprising an electrosurgical energy source in electrical communication with the solenoid coil.

15. The robotic perforator system of claim 11, wherein the heating shaft is disposed in radially spaced relation with the solenoid coil.

16. The robotic perforator system of claim 11, wherein the tube includes non-conductive material.

17. The robotic perforator system of claim 11, wherein inductive energy from the solenoid coil causes an outer surface of the heating shaft to dissipate the amount of heat sufficient to treat tissue.

18. The robotic perforator system of claim 11, wherein the heating shaft extends to a pointed or sharpened tip.

19. The robotic perforator system of claim 18, wherein the heating shaft defines a distal pocket therein.

20. A hand held perforator, comprising:
a handle assembly;
a tube extending distally from the handle assembly, the tube defining a longitudinal axis and a lumen that extends along the longitudinal axis;
a solenoid coil supported within the tube about the longitudinal axis and defining a central passage therethrough; and
a heating shaft received in the central passage and positioned to inductively couple to the solenoid coil, the heating shaft configured to dissipate an amount of heat sufficient to treat tissue received in the lumen when electrical energy is conducted through the solenoid coil.
